(19) United States
(12) Patent Application Publication
(10) Pub. No.: US 2021/0068884 A1
(43) Pub. Date: Mar. 11, 2021

(54) Electrode for prostate surgery and using method thereof

(71) Applicant: SIMAI CO., LTD., Zhuhai (CN)

(72) Inventors: Liping XIE, Zhuhai (CN); Min LIN, Zhuhai (CN)

(21) Appl. No.: 16/932,806

(22) Filed: Jul. 19, 2020

(30) Foreign Application Priority Data
Sep. 5, 2019 (CN) 201921470813.1
Jan. 8, 2020 (CN) 202010017949.8

(51) Int. Cl. A61B 18/00

(52) U.S. Cl. CPC A61B 18/00; A61B 2018/00625; A61B 2018/00607; A61B 2018/00547

(57) ABSTRACT

The invention provides an electrode for a prostate surgery and a using method thereof, and belongs to the technical field of medical apparatus and instruments. The electrode includes an electrode body, and the electrode body is provided with a concave arc surface and a convex arc surface; the electrode body includes a base body, an anterior resecting portion and a posterior resecting portion. The electrode has functions of being enucleatable and resectable, and also has a function of enlarging a hemostatic area, and is capable of improving the hemostatic effect and improving the hemostatic efficiency, thereby the efficiency of the surgery may be improved, the operation difficulty and risk of the surgery are reduced, the bleeding and injury of a patient during the prostate surgery are also reduced at the same time, the safety and success rate of the prostate surgery are improved, and the patients are benefited.

### TECHNICAL FIELD

The invention relates to the technical field of medical apparatus and instruments, and in particular to an electrode for a prostate surgery and a using method thereof.

### BACKGROUND

Along with the continuous improvement of living standards and the advent of an aging society, the number of patients with benign prostatic hyperplasia is greatly increased, a prostate resection becomes a main surgery of urology, and a transurethral resection of prostate (TURP) is always recognized as a golden standard of treating the benign prostatic hyperplasia by urologists, herein, the benign prostatic hyperplasia is a disease caused by the effect of human body factors, such as imbalance of sex hormones, causing glandular connective tissues and smooth muscle tissues of a median lobe or an inverted lobe under a posterior urethral mucosa to be gradually proliferated, forming multiple globular nodules, and enabling a urethra, a bladder and a kidney to occur a series of function disorder. However, a traditional electroscission has many problems, such as much intraoperative bleeding, poor surgical field of vision, easy mis-resection and incomplete tissue enucleation, but the above problems are solved very well by the invention of prostate enucleation, it is very, popular to doctors, and is learned in succession. In the most of existing prostate enucleations, invisible hard removal is performed on a gland by using a scope lemma, and the removal is completely based on the experience and feeling of the doctor. It is very difficult to learn, and a sheath tube is easily damaged because of careless use, and even a ceramic head at a front end of the sheath tube is broken or fallen off, so the safety of the prostate enucleation is seriously affected, and even the failure of the prostate enucleation is caused.

### SUMMARY

In order to solve the problems in an existing technology, the invention aims to provide an electrode for a prostate surgery. The electrode, in a using process of the prostate surgery, has functions of being enucleatable and resectable, and also has a function of enlarging a hemostatic area, and is capable of improving the hemostatic effect and improving the hemostatic efficiency, thereby the efficiency of the prostate surgery may be improved, the operation difficulty and risk of the surgery are reduced, the bleeding and injury of a patient during the prostate surgery are also reduced at the same time, the safety and success rate of the surgery are improved, and the patients are benefited.

In order to achieve the above, purpose, the invention provides the following technical scheme.

An electrode for a prostate surgery, including an electrode body, herein, the electrode body is an arc-shaped sheet structure and may be matched with the surface of a prostate tissue Bayer, and the electrode body is provided with a concave arc surface and a convex arc surface; the electrode body includes a base body, an anterior resecting portion and a posterior resecting portion; the anterior resecting portion is forwards protruded from the base body, and the anterior resecting portion is arc-shaped; and the posterior resecting portion is backwards protruded from the base body, the posterior resecting portion is arc-shaped, in enucleating and resecting processes of the TURF, the convex arc surface may also be used as a component for performing electrocoagulation hemostasis on a place to be resected, namely an electrocoagulation hemostasis part.

Further, the posterior resecting portion is backwards extended from the base body and is opposite to an extending direction of the anterior resecting portion.

Further, a length of the anterior resecting portion forwards protruded from the base body is greater than a length of the posterior resecting portion backwards protruded from the base body.

Further, the length of the anterior resecting portion forwards protruded from the base body is 0.35 mm-0.85 mm.

Further, the length of the posterior resecting portion backwards protruded from the base body is 0.2 mm-1.0 mm.

Further, a height of the electrode body is 1.6 mm-3.1 mm, and a distance between two ends of the electrode body is 3.5 mm-5.5 mm.

Further, a radian of the convex arc surface is 11π/18˜2π/3.

Further, an outer edge of the anterior resecting portion and, an, outer edge of the posterior resecting portion are arc surfaces, and radiuses of the two parties are 0.05 mm-0.3 mm.

Another purpose of the invention is to provide a using method of the above electrode, the using method includes the following steps.

S1: a median lobe of prostate is treated, a hyperplasia tissue of the median lobe of prostate is removed by using the anterior resecting portion of the electrode, and in a peeling process, the electrocoagulation hemostasis is performed on a place to be resected by the convex arc surface;

S2: bilateral lobes of the prostate are treated, hyperplasia tissues of the bilateral lobes of the prostate are removed by using the anterior resecting portion of the electrode, and in a peeling process, the electrocoagulation hemostasis is performed on a place to be resected by the convex arc surface; and

S3: the hyperplasia tissues of the bilateral lobes of the prostate and the hyperplasia tissues of the bilateral lobes of the prostate are acquired by using the posterior resecting portion of the electrode to back-resect.

The beneficial effect of the invention is as follows.

In a process that the electrode provided by the invention is used to perform the prostate surgery, because the electrode body is the arc-shaped sheet structure and may be matched with the surface of the prostate tissue layer, and an arc protruded portion at the front end thereof forms the anterior resecting portion, so the electrode has a function of enucleating the hyperplasia tissue of the prostate like a shovel electrode while it is forwards pushed, it is more labor-saving, easier, and more thorough to remove the tissue; because an arc protruded portion at the rear end of the electrode body forms the posterior resecting portion, the plough-shaped electrode has a function of resecting like a ring electrode while it is backwards pulled, so that the fine trimming and accurate resecting may be performed on the tissue while the electrode is back-resect; in addition, the convex arc surface of the whole electrode body provides the largest area of a coagulation surface in the enucleating and resecting processes, is capable of blood-coagulating in a surgical process, and has a function of hemostasis, thereby a risk of massive bleeding in the surgery is reduced.

### BRIEF DESCRIPTION OF THE DRAWINGS

FIG. 1 is a top view of an electrode in Embodiment 1;

FIG. 2 is a front view of the electrode in Embodiment 1;

FIG. 3 is a A-A section view in FIG. 2; and

FIG. 4 is a stereostructure schematic diagram of the electrode in Embodiment 1.

In the diagram: Electrode body 10, Concave arc surface 11, Convex arc surface 12, Base body 13, Anterior resecting portion 14, Posterior resecting portion 15, Left end portion 16, Right end portion 17, First electrode connecting portion 20, and Second electrode connecting portion 30.

### DETAILED DESCRIPTION OF THE EMBODIMENTS

Technical schemes in the embodiments of the invention are clearly and completely described below in combination with drawings in the embodiments of the invention, apparently, the embodiment described are only a part of the embodiments of the invention, but not all of the embodiments. Based on the embodiments in the invention, all other embodiments acquired by those of ordinary skill in the art without marking creative work shall fall within a scope of protection of the invention.

### Embodiment 1

The embodiment provides an electrode for a prostate surgery, it is a plough-shaped electrode for a TURP, and used for a plasma resectoscope.

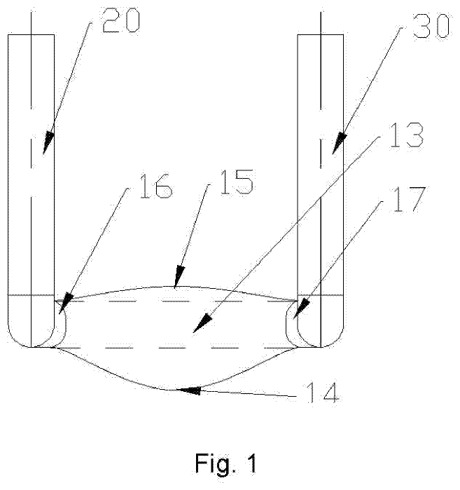

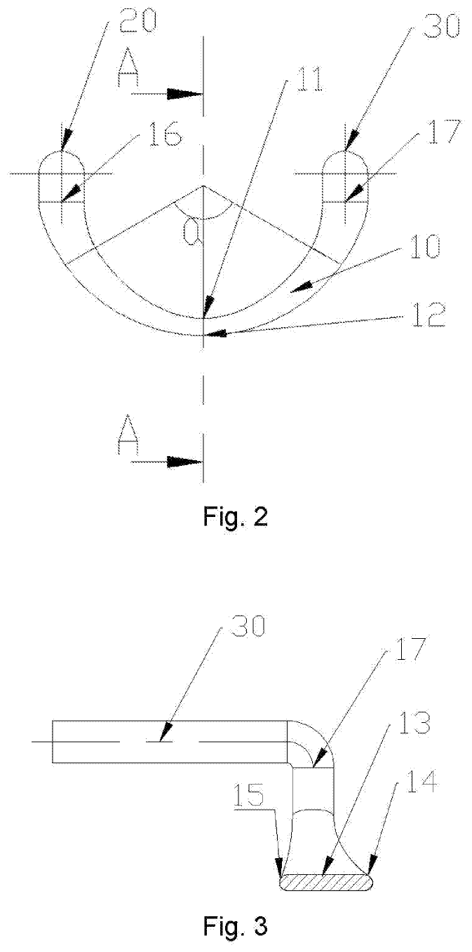

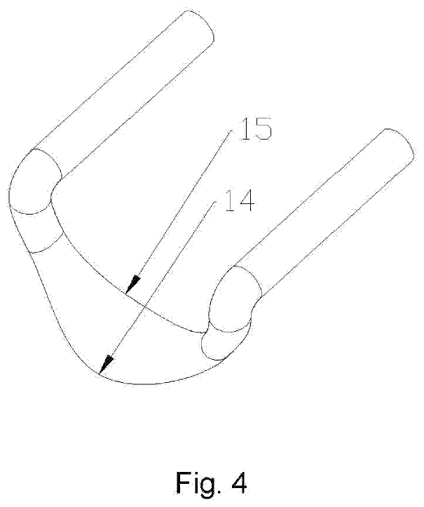

Reference to FIG. 1, FIG. 2 and FIG. 3 specifically, the electrode includes an electrode body 10, the electrode body 10 is an arc-shaped sheet structure and may be matched with the surface of a prostate tissue layer, and the electrode body specifically includes a concave arc surface 11 and a convex arc surface 12, the electrode body 10 may be an axisymmetric structure, such as a tile-shape and, a meniscus-shape. Further, the electrode body 10 includes a base body 13, an anterior resecting, portion 14 and a posterior resecting portion 15; the anterior resecting portion 14 is forwards protruded from the base body 13 and is arc-shaped; the posterior resecting portion 15 is backwards protruded from the base body 13 and is also arc-shaped; the electrode body 10 is the axisymmetric structure, the base body 13, the anterior resecting portion 14 and the posterior resecting portion 15 included in the electrode body 10 are the axisymmetric structures and axes of symmetry of three parties are the same axis of symmetry; in addition, in enucleating and resecting processes of the TURP, the above convex arc surface 12 may also be used as a component for performing electrocoagulation hemostasis on a place to be resected, namely an electrocoagulation hemostasis part.

In the embodiment, during a using process of the prostate surgery, because an arc protruded portion at the front end of the electrode body 10 forms the anterior resecting portion 14, the plough-shaped electrode has a function of enucleating the hyperplasia tissue of the prostate like a shovel electrode while it is forwards pushed, it is more labor-saving, easier, and more thorough to remove the tissue; because an arc protruded portion at the rear end of the electrode body 10 forms the posterior resecting portion 15, the plough-shaped electrode has a function of resecting like a ring electrode (may also be understood as the resecting of the reverse shovel electrode) while it is backwards pulled, so that, the fine trimming and accurate resecting may be performed on the tissue while, the plough-shaped electrode is back-resect; in addition, the convex arc surface 12 of the whole electrode body 10 provides the largest area of a coagulation surface in the enucleating and resecting processes, is capable of blood-coagulating in a surgical process, and has a function of hemostasis, thereby, a risk of massive bleeding in the surgery is reduced.

In the TURP, a tissue of benign prostatic hyperplasia is completely removed through a capsule, so that the surgical wound surface is a complete and smooth prostate capsule, and blood vessels exposed on the capsule are clearly visible, and the electrocoagulation hemostasis may be performed on a place to be resected through the convex arc surface 12, so the contraction and hemostasis of the prostate capsule are more thorough after the surgery, the indwelling catheterization time is shorter, and a patient may even consider not performing bladder irrigation after the surgery.

In the embodiment, reference to FIG. 1 specifically, preferably, the posterior resecting portion 15 is backwards protruded from the base body 13, and is opposite to an extending direction of the anterior resecting portion 14, more further, a length of the anterior resecting portion 14 forwards protruded from the base body 13 is greater than a length of the posterior resecting portion 15 backwards protruded from the base body 13, the plough-shaped electrode is capable of enucleating the tissue like the shovel electrode while it is forwards pushed to be operated, and while the protruded length is larger, it is more labor-saving, easier, and more thorough to remove the tissue; the plough-shaped electrode is capable of resecting like the reverse, shovel electrode while it is backwards pulled to be operated, the protruded length is smaller, the resecting is more accurate, and rapid, the fine trimming of the resecting may be made with a slight convex radian. Specifically, a length from an outer edge of the anterior resecting portion 14 to an outer edge of the posterior resecting portion 15 is 1.0 mm-2.5 mm, so that the convex arc surface 12 as a hemostasis part may provide an enough large coagulation area, herein, the length of the anterior resecting portion 14 forwards protruded from the base body 13 is 0.35 mm-0.85 mm preferably, in addition, the length of the posterior resecting portion 15 backwards protruded from the base body 13 is 0.2 mm-1.0 mm, and the length of the posterior resecting portion 15 backwards protruded from the base body 13 is 0.25 mm preferably; certainly, the above two protruded lengths may also be set to be the same.

Furthermore, in the embodiment, two ends of the electrode body 10 are a left end portion 16 and a right end portion 17 respectively, preferably, a height of the electrode body 10 is 1.6 mm-3.1 mm, a distance between the left end portion 16 and the right end portion 17 of the electrode body 10 is 3.5 mm-5.5 mm, this size setting is to avoid that a size of the electrode body 10 is too large so that occupied space is large, which is not beneficial to the smooth surgery.

Specifically, the electrode of the embodiment further includes two electrode connecting, portions, reference to FIG. 1 specifically, the two electrode connecting portions are a first electrode connecting portion 20 and a second electrode connecting portion 30 respectively, the first electrode connecting portion 20 and the second electrode connecting portion 30 are respectively formed by the left end portion 16 of the electrode body 10 and the right end portion 17 of the electrode body 10 which are backwards extended, the first electrode connecting portion 20 and the second electrode connecting portion 30 are used to be electrically connected with other components of the plasma resectoscope.

In the embodiment, preferably, the radian of the convex arc surface 12 of the electrode body 10 is 11π/18˜2π/3, reference to FIG. 2 specifically, namely a bending angle a of the electrode body 10 is equal to 110°˜120°, and a radius size R of the convex arc surface 12 is equal to 3.0 mm, an insufficient enucleating or resecting surface due to the undersize of the a and R is avoided, an overlarge structure of the whole plough-shaped electrode due to the oversize of the a and R is avoided, overlarge space occupied by the plough-shaped electrode is avoided, and finally the effect of improving surgical efficiency and surgical success rate is achieved.

In the embodiment, preferably, reference to FIG. 2 and FIG. 3 specifically, the outer edge of the anterior resecting portion 14 and the outer edge of the posterior resecting portion 15 are the arc surfaces, radiuses R of two parties are 0.05 mm-0.3 mm, thicknesses of the anterior resecting portion 14 and the posterior resecting portion 15 are gradually increased from the middle to two sides, namely the middle is the thinnest, two ends are the thickest, and it is a smooth transition from the middle to the two ends, a minimum thickness of the middle is set to be 0.2 mm, the insufficient rigidity of the anterior resecting portion 14 and the posterior resecting, portion 15 due to the undersize of the thickness and radius R is avoided, the large strength required by enucleating or resecting due to the oversize of the thickness and radius R is avoided, and finally the effect of guaranteeing the enucleating or resecting accuracy and improving the surgical efficiency and surgical success rate is achieved.

### Embodiment 2

The embodiment is a using method in allusion to the electrode (hereinafter referred to as a plough-shaped electrode) in the above embodiment during a surgical process.

Firstly, the TURP specifically includes the following steps: preoperative preparation work is performed, which specifically includes completing various urinary system examinations before a surgery and the like.

The preoperative treatment work, is performed, preoperative skin preparation is performed, an intestinal tract is prepared, and antibiotics are used for an intraoperative period.

Used surgical equipment and specific parameters, firstly the plough-shaped electrode is assembled into a plasma resectoscope, a plasma resection system, such as Olympus TURis, is used, irrigating solution is used as normal saline, an irrigating height is 60 cm, resection power is 280 W, and electrocoagulation power is 140 W.

A specific surgical method is as follows: firstly, a prostate hyperplasia tissue of a median lobe of prostate is treated, a prostate urethral mucosa is firstly resected at a near end of colliculus seminalis, so a prostate capsule may be seen here, the prostate hyperplasia tissue is peeled off by using an anterior resecting portion 14, electrocoagulation hemostasis is performed while a hemostasis part encounters active bleeding, after the partial tissue is peeled off, the peeled prostate hyperplasia tissue is resected by a posterior resecting portion 15, the peeling is repeated, and the resection is performed to the neck of a bladder, hereafter, prostate hyperplasia tissues of bilateral lobes of the prostate are treated by the same method; thereafter, the posterior resecting portion 15 is used to back-resect to acquire the hyperplasia tissue of the median lobe of the prostate and the hyperplasia tissues of the bilateral lobes of the prostate. In the surgery, attention is paid to the gentle movement, a brute force is not used to cause a perforation of the prostate capsule, the enucleating surgery is terminated while the perforation of the capsule is encountered, and changed to an open surgery or a prostate resection. After the surgery, an, anal finger examination is performed to eliminate the possibility of a rectal injury. At the end of the surgery, the prostate tissues are sucked up by using an irrigator, it is checked again whether there is a prostate tissue in the bladder, whether there is active bleeding on the wound surface, and whether an internal urethral sphincter is injured, a three-cavity catheter is placed to continuously irrigate the bladder after the surgery.

In the embodiment, transurethral vaporization enucleation/prostate resection of the plough-shaped electrode consists of three independent endoscopy procedures, and is used for surgically treating benign prostatic hyperplasia (BPH). Specifically including: 1) a transurethral vaporization enucleation and prostate resection of the plough-shaped electrode, referred to as TVERP, the feature of this technology is that the prostate hyperplasia tissue is almost enucleated and subsequently resected by the plough-shaped electrode; 2) a transurethral vaporization enucleation of the plough-shaped electrode, referred to as TVEP, through such a technology, after the prostate hyperplasia tissue is completely enucleated by the plough-shaped electrode, the hyperplasia tissue may be retrieved by using a tissue crusher, and it saves surgical time while the larger prostate is treated; and 3) an ultrasonic-guided accurate prostate resection, referred to as US-TVERP/TVEP, with this technology, during the TVERP or TVEP surgery, sagittal and lateral structures of the prostate may be displayed by transrectal ultrasound in order to more accurately identify a surgical capsule, an apex and a bladder neck of the prostate, and avoid damage to a urethral sphincter or a bladder wall.

A specific process of the above surgery is as follows.

General anesthesia is performed on a patient, and the patient is placed in a lithotomy position;

firstly, near the plough-shaped electrode, vaporization is performed in a 5 o'clock position of the edge at the near end of the colliculus seminalis, until the surgical capsule is identified by characteristic white circular fibers thereof;

then the surgical capsule in a 7 o'clock position is exposed in the same way;

the vaporization is performed to the bladder neck along the surgical capsule from the 5 o'clock position to the 7 o'clock position;

in a dissection process, the plough-shaped electrode is used for sealing (coagulating) blood vessels and evaporating bonding fibers;

while the separation of the median lobe is performed close to the bladder neck, a left lobe is retrograded along the surgical capsule and enucleated by the plough-shaped electrode in a counterclockwise manner;

then, a right lobe is retrograded along the surgical capsule, and enucleated by the plough-shaped electrode in a clockwise manner; and

then an almost avascular lobe is resected by using the plough-shaped electrode or a resecting ring in a gasification mode, and attention is paid to avoid a perforation of a prostatic vesicle.

The vaporization is performed on the prostate hyperplasia tissue by the plough-shaped in a manner similar to the TVERP, but a detachment process is thoroughly performed through the bladder neck, it is different from the case of incomplete detachment in the TVERP. An enlarged lobe of the prostate is completely enucleated, and then pushed into the bladder. The tissue crusher is used to take out the enucleated prostate tissue during the TVEP.

Before an endoscopy is performed, a dual-wing ultrasonic probe is placed in a rectum, sagittal and cross-sectional images of a surgical area are ultrasonic-displayed, the surgical capsule of the prostate, and the urethral sphincter and the bladder neck around the apex of the prostate may be identified in the ultrasonic images. In addition, in the TVERP or TVEP procedure, a position of a surgical instrument may also be monitored by the ultrasound, such real-time display provides an additional field of view outside an endoscope, it guides the electrode to work along a right plane, directly enter the bladder through the bladder neck, and keep a safe distance from the urethral sphincter. In addition, in a process of retrieving the tissue, it also guides the tip of a silencer away from the bladder wall. Such an ultrasonic guiding technology makes the TVERP and TVEP more accurate, and safer.

Postoperative treatment: indwelling catheterization is used to continuously irrigate the bladder after the surgery, antibiotics and hemostatic drugs are applied, generally, bladder irrigation is stopped after the color of the irrigation is cleared in the second day after surgery, the catheter is pulled out after 3-7 days, the patient is discharged from hospital after urinating conditions and postoperative body temperature are observed to be normal.

Postoperative follow-up visit: after one month after the surgery, a postoperative recovery condition is followed-up, including whether current complication, such as postoperative bleeding and postoperative infection, occurs or not; and the surgical effect may be basically evaluated through 3 months of the follow-up after the surgery, including an international prostate symptom score (IPSS), a quality of life score (QOL), and conditions whether urinary incontinence and urethral stricture and the like occurs or not.

The above embodiments are only used to describe the technical schemes of the invention but not to limit them, the invention is described in detail with reference to the preferable embodiments, those skilled in the art should understand that modifications or equivalent replacements made to the technical schemes of the invention, without departing from purpose and scope of the present technical scheme, shall fall within a scope of the claims of the invention.

What is claimed is:

1. An electrode for a prostate surgery, wherein the electrode comprises an electrode body (10), wherein,
the electrode body (10) is an arc-shaped sheet structure and can be matched with the surface of a prostate tissue layer, and the electrode body (10) is provided with a concave arc surface (11) and a convex arc surface (12); the electrode body (10) comprises a base body (13), an anterior resecting portion (14) and a posterior resecting portion (15);
the anterior resecting portion (14) is forwards protruded from the base body (13), and the anterior resecting portion (14) is arc-shaped;
the posterior resecting portion (15) is backwards protruded from the base body (13), and the posterior resecting portion (15) is arc-shaped; and
the convex arc surface (12) is an electrocoagulation hemostasis part.

2. The electrode for the prostate surgery as claimed in claim 1, wherein the posterior resecting portion (15) is backwards extended from the base body (13) and is opposite to an extending direction of the anterior resecting portion (14).

3. The electrode for the prostate surgery as claimed in claim 2, wherein a length of the anterior resecting portion (14) forwards protruded from the base body (13) is greater than a length of the posterior resecting portion (15) backwards protruded from the base body (13).

4. The electrode for the prostate surgery as claimed in claim 3, wherein the length of the anterior resecting portion (14) forwards protruded from the base body (13) is 0.35 mm-0.85 mm.

5. The electrode for the prostate surgery as claimed in claim 4, wherein the length of the posterior resecting portion (15) backwards protruded from the base body (13) is 0.2 mm-1.0 mm.

6. The electrode for the prostate surgery as claimed in claim 1, wherein a height of the electrode body (10) is 1.6 mm-3.1 mm, and a distance between two ends of the electrode body (10) is 3.5 mm-5.5 mm.

7. The electrode for the prostate surgery as claimed in claim 6, wherein a radian of the convex arc surface (12) is 11π/18−2π/3.

8. The electrode for the prostate surgery as claimed in claim 7, wherein an outer edge of the anterior resecting portion (14) and an outer edge of the posterior resecting portion (15) are arc surfaces, and radiuses of the two parties are 0.05 mm-0.3 mm.

9. A using method of the electrode as claimed in claim 1, wherein the using method comprises the following steps:
S1: treating a median lobe of prostate, and enucleating a hyperplasia tissue of the median lobe of prostate by using the anterior resecting portion (14) of the electrode, wherein in a peeling process, the electrocoagulation hemostasis is performed on a place to be resected by the convex arc surface (12);
S2: treating bilateral lobes of the prostate, and enucleating hyperplasia tissues of the bilateral lobes of the prostate by using the anterior resecting portion (14) of the electrode, wherein in a peeling process, the electrocoagulation hemostasis is performed on a place to be resected by the convex arc surface (12); and
S3: requiring the hyperplasia tissues of the bilateral lobes of the prostate and the hyperplasia tissues of the bilateral lobes of the prostate by using the posterior resecting portion (15) of the electrode to back-resect.
